(19) United States
(12) Patent Application Publication
(10) Pub. No.: US 2017/0224664 A1
(43) Pub. Date: Aug. 10, 2017

(54) POWDER ORAL SUSPENSION FORMULATIONS OF ANTIBACTERIAL AGENTS

(71) Applicant: Cempra Pharmaceuticals, Inc., Chapel Hill, NC (US)

(72) Inventors: David Eugene PEREIRA, Apex, NC (US); Shingai MAJURU, Mebane, NC (US); Prabhavathi FERNANDES, Chapel Hill, NC (US)

(21) Appl. No.: 15/501,516

(22) PCT Filed: Aug. 5, 2015
(86) PCT No.: PCT/US15/43774

§ 371 Date: Feb. 3, 2017

Related U.S. Application Data
(60) Provisional application No. 62/033,601, filed on Aug. 5, 2014.
(60) Provisional application No. 62/173,609, filed on Jun. 10, 2015.

(51) Int. Cl. A61K 31/424; A61K 9/10; A61K 9/14; A61K 9/00

(52) U.S. Cl. CPC A61K 31/424; A61K 9/0095; A61K 9/10; A61K 9/0053; A61K 9/145

(57) ABSTRACT

Powder oral suspension formulations of antibacterial compounds are described herein. In addition, reconstitutable powders of antibacterial compounds and oral suspension formulations thereof are described herein.

### CROSS-REFERENCE TO RELATED APPLICATIONS

This application claims the benefit under 35 U.S.C. §119(e) of U.S. Provisional Application Nos. 62/033,601, filed Aug. 5, 2014, and 62/173,609, filed Jun. 10, 2015, the disclosures of each of which are incorporated herein by reference.

### TECHNICAL FIELD

The invention described herein pertains to powder oral suspension formulations of antibacterial compounds. In particular, the invention described herein pertains to reconstitutable powders of antibacterial compounds and oral suspension formulations thereof.

### BACKGROUND AND SUMMARY OF THE INVENTION

For many years, there has been a long-felt need for an oral suspension formulation for treating bacterial infections. It is well-understood that pediatric indications rely on such formulations for the effective treatment of bacterial infections in children and infants. In addition, other patients, such a geriatric patients, who cannot or will not swallow tablets or capsules, especially when they are above certain dimensional limits, also rely on such formulations for the effective treatment of bacterial infections. However, such formulations often suffer from poor patient compliance due to the undesirable taste characteristics and attributes that are often associated with antibacterial agents. For example, bitterness is a common undesirable taste characteristics. Bitterness not only surfaces in the initial dosing of the antibacterial agent, but also in a latent bitterness effect where the antibacterial agent distributes to the saliva and returns to the mouth. Moreover, latent bitterness may be generally more prolonged than the bitterness associated with initial dosing, and tracks the pharmacokinetics and pharmacodynamics of the antibacterial agent as a consequence of the antibacterial agent returning to the taste receptors in the oral cavity during biodistribution.

It has been reported that erythromycin and other macrolide compounds are substantially bitter to the taste, and that the bitterness of those compounds limits or precludes their use in liquid oral formulations or oral suspension formulations.

Moreover, second generation macrolides, such as clarithromycin, are reportedly as much as 10 times more bitter than erythromycin. In addition, third generation macrolides, such as cethromycin are reportedly as much as about 100 times more bitter than erythromycin.

Without being bound by theory, it is believed herein that the increased bitterness observed for clarithromycin and cethromycin may be related to the modification in each of those compounds at the C6 hydroxy group. In particular, both erythromycin and azithromycin each retains an unmodified C6 hydroxy. In contrast, clarithromycin includes a modified C6 hydroxyl in the form of a methyl ether, and cethromycin includes a modified C6 hydroxyl in the form of the much more sterically demanding quinolinylpropenyl ether. It has been reported that the modified C6 hydroxy group improves both the activity and the stability of macrolide antibacterial compounds. Therefore, though there is a continuing need to develop oral liquid formulations and oral suspension formulations, especially for treatment of pediatric infections, the foregoing observations discourage the use of such second and third generation macrolides because of the presumed unavoidable and unacceptable increase in bitterness.

There continues to be a need to solve the problem of providing macrolide antibacterial agents in oral suspension formulations to ensure good pediatric and other patient compliance.

It has been unexpectedly discovered that oral suspension formulations of the compounds described herein are suitable for treating bacterial infections. In one illustrative and non-limiting embodiment of the invention described herein, compositions, formulations, kits, uses, and methods are described herein that include one more compounds of formula I:

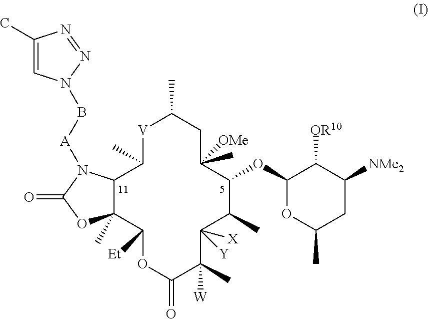

and/or salts or hydrates thereof, and combinations thereof, wherein:

R¹⁰ is hydrogen or acyl;

X is H; and Y is OR⁷; where R⁷ is a monosaccharide or disaccharide, alkyl, heteroalkyl, cycloalkyl, cycloheteroalkyl, aryl, heteroaryl, acyl, or C(O)—NR⁸R⁹, where R⁸ and R⁹ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl, or R⁸ and R⁹ are taken together with the attached nitrogen to form a heterocycle; or X and Y are taken together with the attached carbon to form carbonyl;

V is C(O), C(═NR¹¹), CH(NR¹², R¹³), or N(R¹⁴)CH₂; where R¹¹ is hydroxy or alkoxy, R¹² and R¹³ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl; and R¹⁴ is hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl;

W is H, F, Cl, Br, I, or OH;

A is CH₂, C(O), C(O)O, C(O)NH, S(O)₂, S(O)₂NH, C(O)NHS(O)₂;

B is C₀-C₁₀ alkylene, C₂-C₁₀ alkenylene, or C₂-C₁₀ alkynylene; and

C is hydrogen, hydroxy, acyl, acyloxy, sulfonyl, ureido, or carbamoyl, or alkyl, heteroalkyl, cycloalkyl, cycloheteroalkyl, aryl, heteroaryl, arylalkyl, or heteroarylalkyl, each of which is optionally substituted.

In another embodiment, pharmaceutical compositions containing one or more of the compounds are also described herein. In one aspect, the compositions include a therapeutically effective amount of the one or more compounds for treating a host animal with a bacterial infection. It is to be understood that the compositions may include other components and/or ingredients, including, but not limited to, other therapeutically active compounds, and/or one or more carriers, diluents, excipients, and the like, and combinations thereof. In another embodiment, methods for using the compounds and pharmaceutical compositions for treating host animals with bacterial infection are also described herein. In one aspect, the methods include the step of administering one or more of the compounds and/or compositions described herein to a host animal with bacterial infection. In another aspect, the methods include administering a therapeutically effective amount of the one or more compounds and/or compositions described herein for treating host animals with bacterial infection. In another embodiment, uses of the compounds and compositions in the manufacture of a medicament for treating host animals with bacterial infection are also described herein. In one aspect, the medicaments include a therapeutically effective amount of the one or more compounds and/or compositions for treating a host animal with bacterial infection.

It is to be understood herein that the compounds described herein may be used alone or in combination with other compounds useful for treating bacterial infection, including those compounds that may be therapeutically effective by the same or different modes of action. In addition, it is to be understood herein that the compounds described herein may be used in combination with other compounds that are administered to treat other symptoms of bacterial infection, such as compounds administered to treat pain, and the like.

### BRIEF DESCRIPTION OF THE DRAWINGS

FIG. 1 shows the Time/Intensity Profile of the control formulation (unflavored/unsweetened). Flavors: (a) bitter, (b) green stemmy, (c) moldy cardboard, and (d) tannin mouthfeel. As shown, the control formulation has a strong intensity bitterness that remains patient-perceptible (≧1) for at least 30 minutes in the aftertaste. The aromatic off-notes and tannin mouthfeel also linger at patient-perceptible levels for about 15 minutes in the aftertaste.

FIG. 2 shows the Time/Intensity Profile of POS Formulation 1 (strawberry-flavored/binary sweetener). Flavors: (a) bitter, (b) sweet, (c) strawberry, (d) green stemmy, and (e) moldy cardboard.

FIG. 3 shows the Time/Intensity Profile of POS Formulation 2 (banana-flavored/binary sweetener). Flavors: (a) bitter, (b) sweet, (c) banana, (d) green stemmy, and (e) moldy cardboard.

FIG. 4 shows the Time/Intensity Profile of POS Formulation 3 (strawberry-flavored/sweetened with acesulfame potassium (Ace-K). Flavors: (a) bitter, (b) sweet, (c) strawberry, (d) green stemmy, and (e) moldy cardboard.

FIG. 5A shows the Time/Intensity Profile of POS Formulation 11 (cherry/aspartame) at Day 1. Flavors: (a) bitter, (b) sweet, (c) cherry, and (d) green stemmy. As shown, freshly constituted Formulation 11 provides good coverage of the API bitterness initially. The bitterness of the API is at or below patient-perceptible levels (≦1) overall.

FIG. 5B shows the Time/Intensity Profile of POS Formulation 11 (cherry/aspartame) at Day 7. Flavors: (a) bitter, (b) sweet, (c) cherry, and (d) green stemmy. As shown, formulation 11 provides good bitterness coverage, but may be patient-perceptible (>1) for about 15 minutes in the aftertaste.

FIG. 6A shows the Time/Intensity Profile of POS Formulation 12 (cherry/sucralose) at Day 1. Flavors: (a) bitter, (b) sweet, and (c) cherry. As shown, freshly constituted formulation 12 provides good coverage of the API bitterness initially and throughout the aftertaste with a bitterness at or below patient-perceptible levels (≦1) overall.

FIG. 6B shows the Time/Intensity Profile of POS Formulation 12 (cherry/sucralose) at Day 7. Flavors: (a) bitter, (b) sweet, and (c) cherry. As shown, after 7 days storage at room temperature, formulation 12 provides good coverage of the API bitterness initially and throughout the aftertaste with bitterness at or below patient-perceptible levels (≦1) overall.

FIG. 7A shows the Time/Intensity Profile of POS Formulation 14 (bubblegum/sucralose) at Day 1. Flavors: (a) bitter, (b) sweet, and (c) bubblegum. As shown, freshly constituted formulation 14 provides good coverage of the API bitterness initially and throughout the aftertaste with a bitterness that is at or below patient-perceptible levels (≦1) overall.

FIG. 7B shows the Time/Intensity Profile of POS Formulation 14 (bubblegum/sucralose) at Day 7. Flavors: (a) bitter, (b) sweet, and (c) bubblegum. As shown in FIG. 86, there are no major differences in flavor quality of formulation 14 between the initial and 7-day evaluation periods.

### DETAILED DESCRIPTION

In one illustrative embodiment, the compositions, formulations, kits, uses, and methods described herein include one more compounds of formula I:

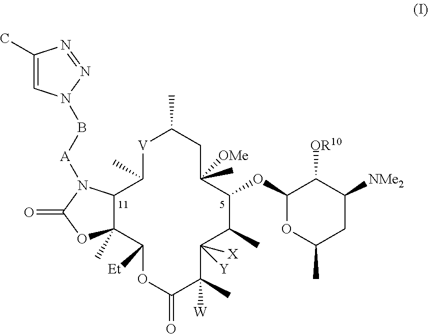

and/or salts or hydrates thereof, and combinations thereof, wherein:

R¹⁰ is hydrogen or acyl;

X is H; and Y is OR⁷; where R⁷ is a monosaccharide or disaccharide, alkyl, heteroalkyl, cycloalkyl, cycloheteroalkyl, aryl, heteroaryl, acyl, or C(O)—NR⁸R⁹, where R⁸ and R⁹ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl, or R⁸ and R⁹ are taken together with the attached nitrogen to form a heterocycle; or X and Y are taken together with the attached carbon to form carbonyl;

V is C(O), C(═NR¹¹), CH(NR¹², R¹³), or N(R¹⁴)CH₂; where R¹¹ is hydroxy or alkoxy, R¹² and R¹³ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl; and R¹⁴ is hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl;

W is H, F, Cl, Br, I, or OH;

A is CH₂, C(O), C(O)O, C(O)NH, S(O)₂, S(O)₂NH, C(O)NHS(O)₂;

B is C₀-C₁₀ alkylene, C₂-C₁₀ alkenylene, or C₂-C₁₀ alkynylene; and

C is hydrogen, hydroxy, acyl, acyloxy, sulfonyl, ureido, or carbamoyl, or alkyl, heteroalkyl, cycloalkyl, cycloheteroalkyl, aryl, heteroaryl, arylalkyl, or heteroarylalkyl, each of which is optionally substituted.

In another embodiment, illustrative triazole-containing ketolide antibiotics include compounds described in WO 2004/080391, and related compounds. Further illustrative triazole-containing ketolide antibiotics include one or more compounds of the formula:

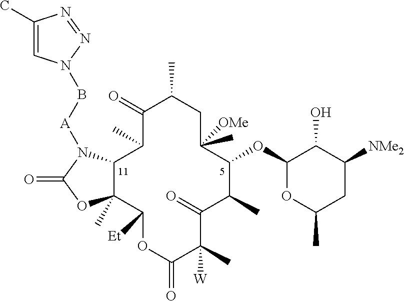

and salts, and hydrates thereof.

Further illustrative triazole-containing ketolide antibiotics include the fluoroketolide compound solithromycin (SOL), Chemical Abstracts Registry Number 760981-83-7, and having the following structure:

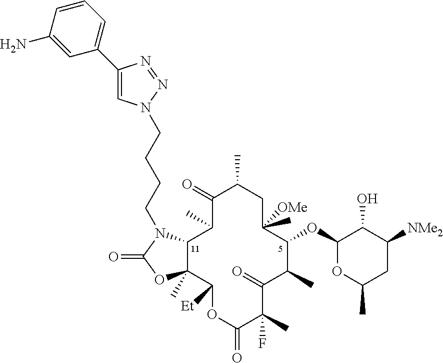 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C]

and salts, hydrates, solvates, and esters thereof. SOL is also described in international patent application, publication number WO 2004/080391. SOL is also known as CEM-101 and as OP-1068. The preparation of SOL and related compounds is described in WO 2009/055557. The disclosures of each of the foregoing publications, and each additional publication cited herein are incorporated herein by reference.

It has been unexpectedly discovered herein that the compounds described herein are less bitter. That discovery is surprising given that the compounds described herein also include C6 modified hydroxy groups. Moreover, that correspondingly higher bitterness threshold is observed both in the initial dosing of the compounds and in the latent bitterness that is observed post-administration when the compounds biodistribute to the saliva.

Without being bound by theory, it is believed herein that the presence of the 1,2.3-triazole in the compounds described herein is at least partly responsible for the observed and surprising decrease in bitterness. It is also believed herein that the presence of the 3-keto group in a subset of the compounds described herein is at least partly responsible for the observed and surprising decrease in bitterness.

The compounds described herein may contain one or more chiral centers, or may otherwise be capable of existing as multiple stereoisomers. It is to be understood that in one embodiment, the invention described herein is not limited to any particular sterochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like Illustratively, such particularly limited length alkyl groups, including C₁-C₈, C₁-C₆, and C₁-C₄, and C₂-C₈, C₂-C₆, and C₂-C₄, and the like may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like Illustratively, such particularly limited length alkenyl and/or alkynyl groups, including C₂-C₈, C₂-C₆, and C₂-C₄, and C₃-C₈, C₃-C₆, and C₃-C₄, and the like may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like. Illustratively, such particularly limited length alkylene groups, including C₁-C₈, C₁-C₆, and C₁-C₄, and C₂-C₈, C₂-C₆, and C₂-C₄, and the like may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like. Illustratively, such particularly limited length alkenylene and/or alkynylene groups, including C₂-C₈, C₂-C₆, and C₂-C₄, and C₃-C₈, C₃-C₆, and C₃-C₄, and the like may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

As used herein, the term “solvates” refers to compounds described herein complexed with a solvent molecule. It is appreciated that compounds described herein may form such complexes with solvents by simply mixing the compounds with a solvent, or dissolving the compounds in a solvent. It is appreciated that where the compounds are to be used as pharmaceuticals, such solvents are pharmaceutically acceptable solvents. It is further appreciated that where the compounds are to be used as pharmaceuticals, the relative amount of solvent that forms the solvate should be less than established guidelines for such pharmaceutical uses, such as less than International Conference on Harmonization (ICH) Guidelines. It is to be understood that the solvates may be isolated from excess solvent by evaporation, precipitation, and/or crystallization. In some embodiments, the solvates are amorphous, and in other embodiments, the solvates are crystalline.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

A wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

Several illustrative embodiments of the invention are described by the following clauses:

A composition in the form of a reconstituable powder for oral suspension, the composition comprising one or more compounds of the formula

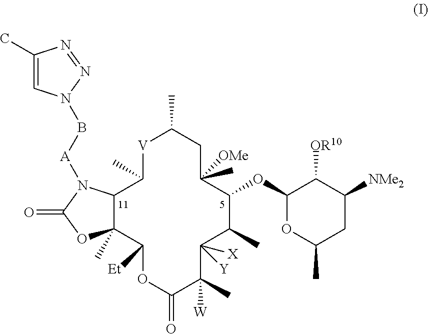

or salts thereof, or hydrates thereof, or combinations thereof, wherein:

R¹⁰ is hydrogen or acyl;

X is H; and Y is OR⁷; where R⁷ is a monosaccharide or disaccharide, alkyl, heteroalkyl, cycloalkyl, cycloheteroalkyl, aryl, heteroaryl, acyl, or C(O)—NR⁸R⁹, where R⁸ and R⁹ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl, or R⁸ and R⁹ are taken together with the attached nitrogen to form a heterocycle; or X and Y are taken together with the attached carbon to form carbonyl;

V is C(O), C(═NR¹¹), CH(NR¹², R¹³), or N(R¹⁴)CH₂; where R¹¹ is hydroxy or alkoxy, R¹² and R¹³ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl; and R¹⁴ is hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl;

W is H, F, Cl, Br, I, or OH;

A is CH₂, C(O), C(O)O, C(O)NH, S(O)₂, S(O)₂NH, C(O)NHS(O)₂;

B is C₀-C₁₀ alkylene, C₂-C₁₀ alkenylene, or C₂-C₁₀ alkynylene; and

C is hydrogen, hydroxy, acyl, acyloxy, sulfonyl, ureido, or carbamoyl, or alkyl, heteroalkyl, cycloalkyl, cycloheteroalkyl, aryl, heteroaryl, arylalkyl, or heteroarylalkyl, each of which is optionally substituted.

The composition of the previous clause wherein R¹⁰ is hydrogen.

The composition of any one of the previous clauses wherein X and Y are taken together with the attached carbon to form carbonyl.

The composition of any one of the previous clauses wherein V is C(O).

The composition of any one of the previous clauses wherein the compound is of the formula

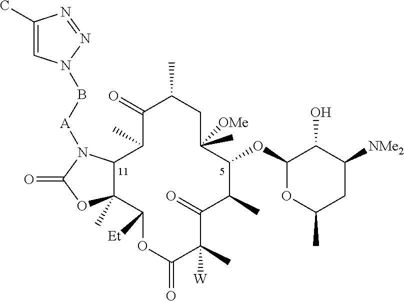

or a salt or a hydrate thereof.

The composition of any one of the previous clauses wherein W is H or F.

The composition of any one of the previous clauses wherein W is F.

The composition of any one of the previous clauses wherein A is CH₂.

The composition of any one of the previous clauses wherein B is (CH₂)ₙ where n is an integer ranging from 0-10, or where n is an integer ranging from 2-6, or where n is an integer ranging from 2-4, or where n is 3.

The composition of any one of the previous clauses wherein C is hydrogen, or alkyl, cycloalkyl, aryl, heteroaryl, arylalkyl, or heteroarylalkyl, each of which is optionally substituted.

The composition of any one of the previous clauses wherein C is alkyl, cycloalkyl, aryl, heteroaryl, arylalkyl, or heteroarylalkyl, each of which is optionally substituted.

The composition of any one of the previous clauses wherein C is aryl, heteroaryl, arylalkyl, or heteroarylalkyl, each of which is optionally substituted.

The composition of any one of the previous clauses wherein C is aryl or heteroarylalkyl, each of which is optionally substituted.

The composition of any one of the previous clauses wherein C is optionally substituted aryl.

The composition of any one of the previous clauses wherein C is aminoaryl.

The composition of any one of the previous clauses wherein C is aminophenyl.

The composition of any one of the previous clauses wherein C is 3-aminophenyl. The composition of any one of the previous clauses wherein the compound is of the formula

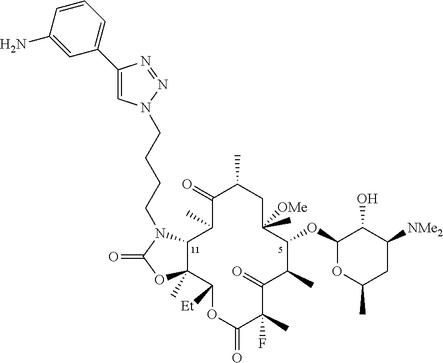 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C]

or a salt or a hydrate thereof.

The composition of any one of the previous clauses wherein the compound is of the formula

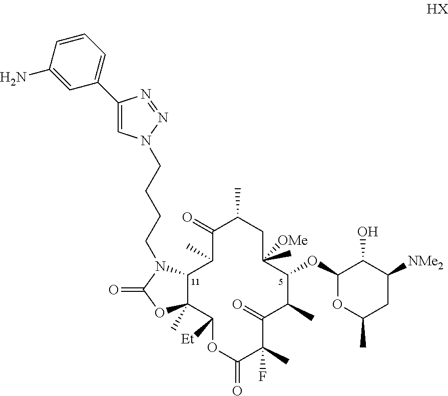 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C]

where HX is a pharmaceutically acceptable acid.

The composition of any one of the previous clauses wherein the compound is of the formula

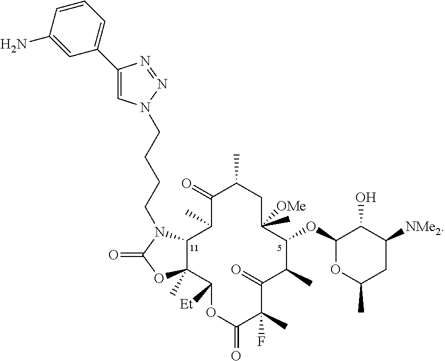 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C]

A kit comprising the composition of any one of the previous clauses and instructions for reconstituting the composition to prepare an oral suspension formulation; and optionally a container for reconstituting.

A pharmaceutical formulation comprising the composition of any one of the previous clauses describing compositions.

The formulation of any one of the previous formulation clauses further comprising a liquid carrier.

The formulation of any one of the previous formulation clauses further comprising a water.

The formulation of any one of the previous formulation clauses configured for oral administration.

The formulation of any one of the previous formulation clauses in the form of a suspension.

The formulation of any one of the previous formulation clauses wherein the bitterness is at a threshold index of about 2 or less, about 1.5 or less, or about 1 or less.

The formulation of any one of the previous formulation clauses wherein the compound is soluble in the formulation at a level of about 1 mg/mL or less, about 0.8 mg/mL or less, about 0.5 mg/mL or less, about 0.3 mg/mL or less, about 0.1 mg/mL or less, or about 0.05 mg/mL or less.

The formulation of any one of the previous formulation clauses wherein the compound is substantially insoluble in the formulation.

The composition, kit, or formulation of any one of the preceding clauses further comprising an excipient capable of lowering the bitterness threshold, the breakthrough bitterness, and/or the bitter flavor perception.

The composition or kit of any one of the preceding clauses capable of being reconstituted in about 15 seconds or less, or about 10 seconds or less.

The composition, kit, or formulation of any one of the preceding clauses free of or substantially free of a reducing sugar.

The composition, kit, or formulation of any one of the preceding clauses wherein the compound is delivered at a dose of about 800 mg.

The composition, kit, or formulation of any one of the preceding clauses wherein the compound is delivered at a dose of about 600 mg.

The composition, kit, or formulation of any one of the preceding clauses wherein the compound is delivered at a dose of about 400 mg.

The composition, kit, or formulation of any one of the preceding clauses wherein the compound is delivered at a dose of about 200 mg.

The composition, kit, or formulation of any one of the preceding clauses further comprising one or more pharmaceutically acceptable constituents selected from the group consisting of suspending agents, sweeteners, preservatives, surfactants, flavoring agents, and combinations thereof.

The composition, kit, or formulation of any one of the preceding clauses further comprising a binary sweetener.

The composition, kit, or formulation of any one of the preceding clauses further comprising a binary sweetener comprising a bulk sweetener and a high intensity sweetener.

The composition, kit, or formulation of any one of the preceding clauses wherein the sweetener comprises sucrose and aspartame.

The composition, kit, or formulation of any one of the preceding clauses wherein the sweetener comprises mono-ammonium glycyrrhizinate or a salt thereof.

It has been discovered herein that the solubility of the compounds described herein generally decreases with increasing pH. For example, SOL has a solubility of 68 mg/mL at pH 5.7, 0.86 mg/mL at pH 6.2, and 0.07 mg/mL at pH 7.5. It has also been discovered herein that at certain pH levels, a stable aqueous suspension of the compounds may be prepared. That suspension may also be prepared from a reconstitutable powder for oral suspension (POS). However, it has also been discovered that at certain limiting pH levels, a threshold amount of the compounds described herein are in solution and therefore such an oral suspension may have a bitter taste that compromises the efficacy thereof, such as due to poor patient compliance.

In another embodiment, described herein are reconstitutable powder for oral suspension (POS) formulations, and the corresponding suspension formulations, that provide a below bitterness threshold amount of active compound in solution. In another embodiment, described herein are reconstitutable POS formulations, and the corresponding suspension formulations, that include a flavor or other bitterness masking agent.

Illustratively, the POS and suspension formulations described herein include a buffer, such as, but not limited to, tribasic sodium phosphate, tribasic sodium phosphate anhydrous, tribasic potassium phosphate, tribasic potassium phosphate anhydrous, alkaline borate, sodium bicarbonate, sodium carbonate, potassium bicarbonate, potassium carbonate, and the like.

In another embodiment, the reconstituted POS formulation described herein have a pH in the range from about 7 to about 10, from about 7 to about 9.5, from about 7 to about 9, from about 7 to about 8.5, from about 7 to about 8.

In another embodiment, the reconstituted POS formulation described herein have a pH in the range from about 7.5 to about 10, from about 7.5 to about 9.5, from about 7.5 to about 9, from about 7.5 to about 8.5, from about 7.5 to about 8.

In another embodiment, the reconstituted POS formulation described herein have a pH in the range from about 7.8 to about 10, from about 7.8 to about 9.5, from about 7.8 to about 9, or from about 7.8 to about 8.5.

In another embodiment, the reconstituted POS formulation described herein have a pH in the range from about 8 to about 10, from about 8 to about 9.5, or from about 8 to about 9.

In another embodiment, the reconstituted POS formulation described herein have a pH in the range from about 8.5 to about 10, from about 8.5 to about 9.5, or from about 8.5 to about 9.

In another embodiment, the POS formulation described herein include a buffer capable of maintaining the pH of the reconstituted suspension formulation above about 7, above about 7.5, above about 7.8, above about 8, or above about 8.5.

It has been unexpectedly discovered that the reconstituted suspension formulations described herein exhibit slow equilibrium buffer kinetics. It is appreciated that once reconstituted, the suspension formulations are generally used over a period of about 5-10 and/or 5-14 days. It has been observed that the pH of the suspension formulations decreases over the dosing time. It is also appreciated that, generally, the buffer capacity of a pharmaceutical preparation for in vivo use is not substantially greater than that of the host animal receiving the formulation. In addition, it is also appreciated that, generally, the pH of the buffer of a pharmaceutical preparation for in vivo use is not substantially greater than that of the host animal receiving the formulation. Otherwise, the buffer of the pharmaceutical preparation may overload and/or overwhelm the pH homeostasis of the host animal. Nevertheless, it has been discovered herein that when the suspension formulations described herein include higher pH, higher strength, and/or higher capacity buffers, their performance is improved. Such improvements may include improved stability, and lower breakthrough bitterness thresholds.

In another embodiment, the POS formulations described herein include a buffer capable of maintaining a pH in the range from about 7 to about 10, from about 7 to about 9.5, from about 7 to about 9, from about 7 to about 8.5, from about 7 to about 8 throughout the dosing period, such as 10 days.

In another embodiment, the POS formulations described herein include a buffer capable of maintaining a pH in the range from about 7.5 to about 10, from about 7.5 to about 9.5, from about 7.5 to about 9, from about 7.5 to about 8.5, from about 7.5 to about 8 throughout the dosing period, such as 10 days.

In another embodiment, the POS formulations described herein include a buffer capable of maintaining a pH in the range from about 7.8 to about 10, from about 7.8 to about 9.5, from about 7.8 to about 9, or from about 7.8 to about 8.5 throughout the dosing period, such as 10 days.

In another embodiment, the POS formulations described herein include a buffer capable of maintaining a pH in the range from about 8 to about 10, from about 8 to about 9.5, or from about 8 to about 9 throughout the dosing period, such as 10 days.

In another embodiment, the POS formulations described herein include a buffer capable of maintaining a pH in the range from about 8.5 to about 10, from about 8.5 to about 9.5, or from about 8.5 to about 9 throughout the dosing period, such as 10 days.

In another embodiment, the POS formulation described herein include a buffer capable of maintaining the pH of the reconstituted suspension formulation above about 7, above about 7.5, above about 7.8, above about 8, or above about 8.5 throughout the dosing period, such as 10 days.

In another embodiment, the weight/weight ratio of API/buffer is less than about 70, less than about 50, less than about 30, less than about 20, or less than about 15. In another embodiment, the weight/weight ratio of API/buffer is less than about 12, less than about 10, less than about 9, less than about 8, less than about 7, less than about 6, or less than about 5.

It has been unexpectedly discovered herein that compounds described herein require multi-dimensional flavor masking, including not only the masking of mimetic bitterness, but also latent and lingering bitterness, aromatic off-notes, including soapy, green stemmy, moldy and/or cardboard tastes, and mouthfeel effects, including soapy, tannin, tongue sting, and drying sensations. Described herein are solutions to the problems for each of the foregoing flavor masking dimensions.

It has also been unexpectedly discovered herein that various sweetening agents and various combinations of sweetening agents may not provide in all instances a sufficiently desirable decrease in the residual bitterness of formulations of compounds described herein. It has also been unexpectedly discovered that various sweetening agents and various combinations of sweetening agents, when combined with one or more predetermined additional flavor modifying components, provide improved reductions in bitterness.

In an illustrative embodiment, the addition of sodium chloride for saltiness and a souring agent produced an improved flavor profile of oral suspensions, an in particular for formulations containing a combination of the sweeting agents, such as sucrose and sucralose, sucrose and aspartame, and sucrose, aspartame, and Magnasweet. The combination of salt, sour, and sweet unexpectedly reduced the intensity of the undesirable flavor attributes of the compounds described herein, including bitterness, aromatic off notes and mouthfeel, below the typical patient perception levels (less than 1).

In another embodiment, the reconstitutable powder for oral suspension (POS) formulations, and the corresponding suspension formulations, include one or more sweeteners or sweetening agents. Illustrative sweeteners and sweetening agents include high intensity sweeteners and bulk sweeteners, and combinations thereof. Illustrative high intensity sweeteners include, but are not limited to, acesulfame potassium (Ace-K), sodium saccharin, neotame, aspartame, sucralose, and the like, and combinations thereof. Illustrative bulk sweeteners include, but are not limited to, sucrose, xylitol, erythritol, mannitol, sorbitol, trehalose, powdered hydrogenated maltose starch syrup, and the like, and combinations thereof. In another embodiment, the reconstitutable POS formulations, and the corresponding suspension formulations, include one or more taste modifying agents, such as, but not limited to, mono-ammonium glycyrrhizinate (Magnasweet™), thaumatin (Talin™), and the like.

In another embodiment, the weight/weight ratio of total sweetener/API is greater than about 5, greater than about 6, greater than about 7, greater than about 8, or greater than about 9.

In another embodiment, the high intensity sweetener is present in a range. It has been unexpectedly discovered that the ability of the high intensity sweeteners to mask undesirable taste attributes, such as bitterness, decreases below and above a predetermined range. In one embodiment, the sweetener Ace-K is present in the range from about 0.2% to about 0.6%, or about 0.3% to about 0.5% of the reconstituted oral suspension. In another embodiment, the sweetener sodium saccharin is present in the range from about 0.2% to about 0.6%, or about 0.3% to about 0.5% of the reconstituted oral suspension. In another embodiment, the sweetener neotame is present in the range from about 0.01% to about 0.03%, or about 0.15% to about 0.25% of the reconstituted oral suspension. In another embodiment, the sweetener aspartame is present in the range from about 0.5% to about 0.9%, or about 0.6% to about 0.8% of the reconstituted oral suspension. In another embodiment, the sweetener sucralose is present in the range from about 0.4% to about 0.8%, or about 0.5% to about 0.7% of the reconstituted oral suspension.

In another embodiment, the high intensity sweetener is present in a range. It has been unexpectedly discovered that the ability of the high intensity sweeteners to mask undesirable taste attributes, such as bitterness, decreases below and above a predetermined range. In one embodiment, the ratio of API to sweetener Ace-K is in the range from about 4 to about 8, or about 5 to about 7. In another embodiment, the ratio of API to sweetener sodium saccharin is in the range from about 4 to about 8, or about 5 to about 7. In another embodiment, the ratio of API to sweetener neotame is in the range from about 80 to about 160, or about 100 to about 140. In another embodiment, the ratio of API to sweetener aspartame is in the range from about 2 to about 6, or about 3 to about 5. In another embodiment, the ratio of API to sweetener sucralose is in the range from about 6 to about 10, or about 7 to about 9.

In another embodiment, the bulk sweetener is sucrose. In another embodiment, the sucrose is present at a concentration of about 30 to about 90 g/100 mL of reconstituted oral suspension, about 30 to about 80 g/100 mL of reconstituted oral suspension, about 50 to about 90 g/100 mL of reconstituted oral suspension, about 50 to about 80 g/100 mL of reconstituted oral suspension, or about 60 to about 80 g/100 mL of reconstituted oral suspension.

It has been unexpectedly discovered that combinations of sweetening agents are more effective at reducing bitterness, than single agents, such a sucrose alone, aspartame alone, Ace-K alone, and/or sucralose. It has been unexpectedly discovered that ternary combinations of sweetening agents are not significantly more effective than binary combinations at reducing bitterness below the typical patient perception levels (less than about 1½). In an illustrative embodiment, oral suspensions are described herein that include a mixture of the sweetening agents, such as sucrose and sucralose, sucrose and aspartame, and aspartame and sucralose, and one or more additional flavor modifying components, such as taste modifying agents, salting agents, such as sodium chloride, and/or souring agents.

In an illustrative embodiment, oral suspensions are described herein that include a binary mixture of the sweetening agents comprising sucrose and aspartame. In an illustrative embodiment, oral suspensions are described herein that include a binary mixture of the sweetening agents comprising sucrose and sucralose.

It has been unexpectedly discovered that salting agents, such as sodium chloride and souring agents provide an improved effect with binary sweeteners sucrose and sucralose, than with single agents alone, or other sweetener combinations described herein. Improved effects include reductions in various aspects of bitterness, reductions in various aspects of aromatic off-notes, and reductions in various aspects of mouth-feel characteristics below the typical patient perception levels (less than about 1½). In an illustrative embodiment, oral suspensions are described herein that include a sucrose and sucralose, or sucrose and aspartame, and a salting agent, such as sodium chloride, and/or a souring agent.

It has been unexpectedly discovered that taste modifying agents, such as Magnasweet, provides an improved effect with binary sweeteners aspartame and sucralose, than with single agents alone, or other sweetener combinations described herein. Improved effects include reductions in various aspects of bitterness, reductions in various aspects of aromatic off-notes, and reductions in various aspects of mouth-feel characteristics below the typical patient perception levels (less than about 1½). In an illustrative embodiment, oral suspensions are described herein that include a sucrose and aspartame or sucrose and sucralose, and Magnasweet. In another embodiment, oral suspensions are described herein that include Magnasweet in the range of about 0.005% to about 0.05%, or about 0.008% to about 0.02% of the total reconstituted oral suspension formulation. It was unexpectedly observed that the ability of Magnasweet to extend the and support the sweetener decreased on either side of a predetermined range.

It has also been discovered herein that the amount of certain excipients, such as sucrose, in the suspension formulations described herein affects the suspendability of the compounds described herein. Without being bound by theory, it is believed herein that the changes in density of the suspension formulations caused by those excipients contributes at least in part to the suspendability of the compounds.

It has unexpectedly been discovered that compounds described herein, such as SOL, are unstable in the presence of high relative amounts of sucralose. In another embodiment, the weight/weight ratio of API, such as SOL to sucralose is greater than about 3, greater than about 4, greater than about 5, greater than about 6, greater than about 7, greater than about 8, greater than about 9, or greater than about 10.

Illustratively, the POS and suspension formulations described herein include salty agents such as, but not limited to, NaCl, and the like.

Illustratively, the POS and suspension formulations described herein include one of more souring agents. It has unexpectedly been discovered that compounds described herein, such as SOL, are unstable in the presence of high relative amounts of citric acid. In another embodiment, the weight/weight ratio of API, such as SOL to citric acid is greater than about 50, greater than about 60, greater than about 70, greater than about 80, or greater than about 90. In another embodiment, the POS and suspension formulations described herein are substantially free of, or free of citric acid.

In another embodiment, the reconstitutable powder for oral suspension (POS) formulations, and the corresponding suspension formulations, include one or more flavors or flavoring agents. Illustrative flavors and flavoring agents include, but are not limited to, orange, lemon, lemon-lime, citrus, cherry, bubble gum, strawberry, raspberry, cherry, mixed berry, grape, vanilla, watermelon, pineapple, peach, mango, banana, tropical fruit, fruit punch, tutti-frutti, and the like.

Illustratively, the reconstitutable powder for oral suspension (POS) formulations, and the corresponding suspension formulations, include one or more suspending agents, antifoaming agents, glidants, and/or preservatives.

Illustratively, the POS and suspension formulations described herein include one or more excipients such as, but not limited to, sucrose, xylitol, erythritol, mannitol, sorbitol, powdered hydrogenated starch hydolysate, trehalose, hydroxypropyl cellulose, hypromellose, methylcellulose, aspartame, sucralose, acesulfame potassium, thaumatin, amino methacrylate copolymer, ammoniomethacrylate copolymer Type A, sucrose stearate, glyceryl monostearate, hydrated silicon dioxide, colloidal silicon dioxide, methylparaben, potassium sorbate, xanthan gum, carboxymethylcellulose sodium, and the like.

Illustratively, the reconstitutable powder for oral suspension (POS) formulations, and the corresponding suspension formulations, include one or more suspending agents, antifoaming agents, and/or preservatives.

Illustratively, the POS and suspension formulations described herein include viscosity modifying agents such as, but not limited to, xanthan gum, and the like. It is understood that viscosity modifying agents such as, but not limited to, xanthan gum, and the like may also function as suspending agents and/or suspension stabilizing agents.

Illustratively, the POS and suspension formulations described herein include foam forming modifying agents such as, but not limited to, simethicone, and the like.

Illustratively, the POS and suspension formulations described herein include glidants such as, but not limited to, colloidal silicon dioxide, Aerosol 200, and the like.

Illustratively, the POS and suspension formulations described herein include preservatives such as, but not limited to, potassium sorbate, and the like.

It has also been discovered herein that the particle size of the suspension affects both mouth feel and grittiness. It has been discovered that a small particle size improves both the mouth feel and grittiness components. It has also been discovered herein that a small particle size improves the stability of the suspension. It has also been discovered that a small particle size improves the homogeneity of the suspension. However, it is understood that if the particle size is too small then breakthrough bitterness may result. Accordingly, described herein are optimized particle sizes that maximize the mouth feel, minimize the grittiness, maximize the stability and homogeneity of the suspension, and minimize the potential for breakthrough bitterness resulting from partial dissolution of the compounds described herein in the suspension formulations. Without being bound by theory, it is believed herein that a particle size below a predetermined lower limit provides an unexpectedly high increase in surface area that may cause excess dissolution of the compounds.

In one embodiment, the D90 particle size of the compounds described herein is less than about 300, less than about 275, less than about 250, less than about 225, less than about 200, or less than about 190 μm. In another embodiment, the D90 particle size of the compounds described herein is less than about 150, less than about 135, less than about 125, less than about 120, less than about 115, less than about 110, less than about 105, or less than about 100 μm. It is to be understood that in other embodiments, the D90 particle size of the compounds described herein is in a range set by the selection of any of the upper limits described herein, such as in the range from about 190 to about 300 μm, about 190 to about 275 μm, about 190 to about 250 μm, about 190 to about 225 μm, about 190 to about 200 μm, about 200 to about 300 μm, about 200 to about 275 μm, about 200 to about 250 μm, and all other possible combinations.

In another embodiment, the D50 particle size of the compounds described herein is about 90, about 80, about 70, about 60, about 50, about 40, or about 30 μm. In another embodiment, the D50 particle size of the compounds described herein is about 45, about 40, about 35, about 30, about 25, about 20, or about 15 μm.

In another embodiment, the D10 particle size of the compounds described herein is greater than about 3, greater than about 4, greater than about 5, greater than about 6, greater than about 7, greater than about 8, or greater than about 9 μm. In another embodiment, the D10 particle size of the compounds described herein is greater than about 2, greater than about 3, greater than about 4, greater than about 5, greater than about 6, or greater than about 7 μm.

The effective use of the compounds, compositions, and methods described herein for treating or ameliorating one or more diseases caused by a pathogenic organism using one or more compounds, compositions, kits, or formulations described herein may be based upon animal models, such as murine, canine, porcine, and non-human primate animal models of disease. For example, it is understood that a bacterial infection in humans may be characterized by a loss of function, and/or the development of symptoms, each of which may be elicited in animals, such as mice, and other surrogate test animals, such as those described herein.

The following examples further illustrate specific embodiments of the invention; however, the following illustrative examples should not be interpreted in any way to limit the invention.

### Examples

### Example

A reconstitutable POS formulation is described herein that comprises a compound described herein, sucrose, sucralose, sodium chloride, anhydrous trisodium phosphate, xanthan gum, colloidal silicon dioxide, simethicone, potassium sorbate, and flavor.

### Example

A reconstitutable POS formulation is described herein that comprises a compound described herein, sucrose, aspartame, Magnasweet, anhydrous trisodium phosphate, xanthan gum, colloidal silicon dioxide, simethicone, potassium sorbate, and flavor.

### Example

A reconstitutable POS formulation is described herein that comprises a compound described herein, sucrose, aspartame, acesulfame potassium, anhydrous trisodium phosphate, xanthan gum, colloidal silicon dioxide, simethicone, potassium sorbate, and flavor.

### Example

A reconstitutable formulation is described herein that includes 60.5 g of reconstitutable powder for oral suspension comprising 6.4 g of SOL and excipients selected from sucrose, aspartame, acesulfame potassium, anhydrous trisodium phosphate, xanthan gum, colloidal silicon dioxide, simethicone, potassium sorbate, and natural strawberry flavor. The formulation is reconstituted as follows:

| | | Total volume of | | |
|---|---|---|---|---|
| | | reconstituted | | Volume |
| POS | Water | oral suspension | SOL | containing 400 mg |
| (g) | (mL) | formulation (mL) | Concentration | dose of soli (mL) |
| 60.5 | 70 | 132 | 48.5 mg/mL | 8.25 |

### Example

A reconstitutable POS formulation kit is described herein according to the following.

| | Quantity of powder per bottle | Bottle Description |
|---|---|---|
| SOL POS | 60.5 g | 150 cc HDPE Container |

Plastic closure child resistant 38 mm

Similar kits are prepared using any of the POS formulations described herein.

### Example

A reconstitutable POS formulation is described herein according to the following.

| Ingredient | Function | Formula (% w/v) |
|---|---|---|
| SOL | Active Pharmaceutical | 6.4 |
| | Ingredient | |
| Sucrose | Taste Masking/Sweetener | 45 |
| Aspartame | Taste Masking/Sweetener | 0-1.5 |
| Acesulfame Potassium | Taste Masking/Sweetener | 0.8 |
| Tribasic sodium | pH modifier | 0.1 |
| phosphate anhydrous | | |
| Xanthan gum | Viscosity modifier | 0.15 |
| Colloidal silicon dioxide | Glidant | 0.5 |
| Simethicone | Anti-foaming agent | 0-0.15 |
| Potassium Sorbate | Preservative | 0.2 |
| Flavor | Flavor | 0.5 |

### Example

A reconstitutable POS formulation is described herein according to the following.

| Ingredient | % w/w | Quantity (g in 100 mL water) | |
|---|---|---|---|
| SOL | 10.6 | 6.40 | |
| Sucrose | 82.6 | 50.0 | |
| Aspartame | 2.48 | 1.50 | |
| Acesulfame potassium | 1.32 | 0.80 | |
| Tribasic sodium phosphate | 0.16 | 0.10 | |
| anhydrous | | | |
| Xanthan gum | 0.50 | 0.30 | |
| Colloidal silicon dioxide | 0.83 | 0.50 | |
| Simethicone | 0.33 | 0.20 | |
| Potassium Sorbate | 0.33 | 0.20 | |
| Strawberry Flavor | 0.83 | 0.50 | |
| Total per bottle | 100 | 60.5 | g |

### Example

Reconstitutable POS formulations are described herein comprising the following.

| | Formulation 1 | Formulation 2 | Formulation 3 |
|---|---|---|---|
| Ingredient | (% w/v) | (% w/w) | (% w/v) |
| SOL | 6.4 | 6.4 | 6.4 |
| Sucrose | 50 | 50 | 50 |
| Aspartame | 1.5 | 1.5 | 0 |
| Acesulfame potassium | 0.8 | 0.8 | 0.8 |
| Tribasic sodium | 0.1 | 0.1 | 0.1 |
| phosphate anhydrous | | | |
| Xanthan gum | 0.3 | 0.3 | 0.3 |
| Colloidal silicon | 0.5 | 0.5 | 0.5 |
| dioxide | | | |
| Simethicone | 0.2 | 0.2 | 0 |
| Potassium Sorbate | 0.2 | 0.2 | 0.2 |
| Strawberry Flavor | 0.5 | 0 | 0.5 |
| Banana flavor | 0 | 0.5 | 0 |

### Example

Reconstitutable POS formulations are described herein comprising the following.

| | 2A | | 2B | | 4A | | 4B | |
|---|---|---|---|---|---|---|---|---|
| | % | | % | | % | | % | |
| | w/w | g/batch | w/w | g/batch | w/w | g/batch | w/w | g/batch |
| SOL | 12.8 | 191.4 | 12.8 | 192.6 | 12.8 | 191.8 | 12.8 | 192.2 |
| Sucrose | 79.7 | 1196.2 | 80.2 | 1203.4 | 79.9 | 1198.6 | 80.1 | 1201.0 |
| Aspartame | 3.18 | 47.8 | 3.20 | 48.1 | 3.19 | 47.9 | 3.20 | 48.0 |
| Tribasic Sodium | 1.12 | 16.8 | 1.13 | 16.9 | 1.12 | 16.9 | 1.13 | 16.9 |
| Phosphate, Anhydrous | | | | | | | | |
| Xanthan Gum | 0.60 | 9.87 | 0 | 0 | 0.4 | 6 | 0.2 | 3 |
| Colloidal Silicon | 1.00 | 15.0 | 1.00 | 15.1 | 1.00 | 15.0 | 1.001 | 15.0 |
| Dioxide | | | | | | | | |
| Simethicone | 0.40 | 5.99 | 0.40 | 6.02 | 0.4 | 6 | 0.4 | 6 |
| Potassium Sorbate | 0.40 | 6.0 | 0.40 | 6.0 | 0.40 | 6.0 | 0.40 | 6.0 |
| MagnaSweet | 0.046 | 0.69 | 0.046 | 0.69 | 0.046 | 0.69 | 0.046 | 0.69 |
| Red Dye #40 | 0.029 | 0.44 | 0.029 | 0.44 | 0.029 | 0.44 | 0.029 | 0.44 |
| Artificial Cherry | 0.72 | 10.8 | 0.72 | 10.8 | 0.72 | 10.8 | 0.72 | 10.8 |
| Flavor | | | | | | | | |
| Total | 100 | 15000 | 100 | 1500 | 100 | 1500 | 100 | 1500 |

### Example

Reconstitutable POS formulations are described herein comprising the following.

| | 6A | | 11A | |
|---|---|---|---|---|
| | % w/w | g/batch | % w/w | g/batch |
| SOL | 12.7 | 190.8 | 12.7 | 1267 |
| Sucrose | 79.5 | 1192.2 | 79.2 | 79198 |
| Aspartame | 3.17 | 47.6 | 3.16 | 316 |
| Tribasic Sodium Phosphate, | 1.85 | 27.8 | 2.2 | 220 |
| Anhydrous | | | | |
| Xanthan Gum | 0.60 | 8.94 | 0.59 | 59.4 |
| Colloidal Silicon Dioxide | 0.99 | 14.9 | 0.99 | 99.0 |
| Simethicone | 0.397 | 5.96 | 0.40 | 39.6 |
| Potassium Sorbate | 0.40 | 6.0 | 0.40 | 6.0 |
| MagnaSweet | 0.046 | 0.69 | 0.046 | 4.6 |
| Red Dye #40 | 0.029 | 0.44 | 0.029 | 2.9 |
| Artificial Cherry Flavor | 0.72 | 10.7 | 0.71 | 71.3 |
| Total | 100 | 1500 | 100 | 10000 |

### Example

The following formulations are described for preparing 320 mg/5 mL doses.

| | Formulation | Formulation | Formulation | Formulation |
|---|---|---|---|---|
| | 11 | 12 | 13 | 14 |
| | Batch | Batch | Batch | Batch |
| | Weight (g) | Weight (g) | Weight (g) | Weight (g) |
| SOL | 6.4 | 6.4 | 6.4 | 6.4 |
| Aerosol 200 | 0.5 | 0.5 | 0.5 | 0.5 |
| Sodium Phosphate Tribasic | 0.1 | 0.1 | 0.1 | 0.1 |
| Anhydrous | | | | |
| Potassium Sorbate, NF | 0.2 | 0.2 | 0.2 | 0.2 |
| Magnasweet 100 | 0.023 | — | 0.023 | — |
| Xanthan Gum* | 0.3 | 0.3 | — | — |
| Simethicone | 0.2 | 0.2 | 0.2 | 0.2 |
| Aspartame | 1.6 | — | 1.6 | — |
| Sucrose | 40 | 35 | 40 | 35 |
| Sucralose | — | 0.74 | — | 0.74 |
| Sodium Chloride | — | 0.85 | — | 0.85 |
| Art Cherry Flavor NV- | 0.36 | 0.36 | — | — |
| 20,629 | | | | |
| Art Bubblegum Flavor NV- | — | — | 0.36 | 0.36 |
| 10,506 | | | | |
| Water | qs 100 mL | qs 100 mL | qs 100 mL | qs 100 mL |
| Sodium Phosphate Tribasic | pH to 8.0 | pH to 8.0 | pH to 8.0 | pH to 8.0 |
| Anhydrous | | | | |
| Citric Acid | pH to 8.0 | pH to 8.0 | pH to 8.0 | pH to 8.0 |

### Example

Reconstituted oral suspension formulation. The SOL POS in bottles with the compositions described herein is reconstituted with water to a total volume of 100 mL.

| POS | Water | SOL Concentration After |
|---|---|---|
| (g) | (mL) | Reconstitution (mg/mL) |
| 30.3 | 69.8 | 32 |
| 60.5 | 39.5 | 64 |

| | | | SOL Concentration |
|---|---|---|---|
| POS | Water | Final Volume | After Reconstitution |
| (g) | (mL) | (mL) | (%) |
| 10 | 30 mL | 40 | 3.2 |
| 20 | 18 mL | 40 | 6.4 |

### Example

Reconstituted oral suspension formulation. Compositions described herein are partitioned into HDPE bottles, each containing 20 g of reconstitutable POS. Each 20 g of reconstitutable POS is reconstituted with 40 mL of water to provide an oral suspension formulation with the specified concentration of API. For example, the 6.4% w/v POS Formulation provides a 64 mg/mL oral suspension, and the 3.2% w/v POS Formulation provides a 32 mg/mL oral suspension.

### Example

A preparation of a reconstitutable formulation is described herein, and includes the following steps:

Step 1: Using a graduated cylinder or a syringe measure 70 mL of purified water.

Step 2: Shake one bottle of powder for oral suspension (60.5 g) to loosen the powder, open the bottle and then remove the induction seal liner.

Step 3: Add approximately 45 mL of the water to the powder. Tightly close the HDPE bottle and shake vigorously by hand continuously for 2 minutes.

Step 4: Allow the bottle to sit for about 1 minute.

Step 5: Remove the closure and add the remainder of the water (25 mL). Close the bottle and shake vigorously by hand for 2 minutes.

Step 6: Allow the suspension to sit for at least 12 hours before use

Step 7: After reconstitution 400 mg of SOL is contained in 8.25 mL of the suspension. Swirl (gently shake) before dispensing a dose.

### Example

Reconstitutable POS Formulation Stability Testing. The reconstitutable POS formulations and corresponding suspension formulations described herein are stored at 40° C./75% RH in open containers and at 60° C. in closed containers for 4 weeks. Illustratively, the compound to excipient ratio is 1:4, and the compound is present in the suspension formulation at a concentration of about 32 mg/mL (for example, 40 mg/1.25 mL) and at pH 8. After storage, the test samples are evaluated for compound assay, impurities, appearance, and odor. The POS formulations and corresponding suspension formulations described herein are generally stable and result in a total impurity level of less than about 4%.

### Example

Reconstituted Oral Suspension Stability Testing. The reconstitutable POS formulations described herein are reconstituted by adding water. The 6.4% w/v POS Formulation and the 3.2% w/v POS Formulation result in a viscous, pink, opaque suspension with white specks interspersed throughout the suspension that is homogenous with no visible contaminants. The reconstituted oral suspension formulation is stored at either ambient temperature (RT) or 5° C. The pH and assay are measured periodically. The stability results for Example Formulation 2A are as follows.

| | | | Water | Day 0 | Assay | Day 1 | Assay | Day 5 | Assay | Day 9 | Assay |
|---|---|---|---|---|---|---|---|---|---|---|---|
| Storage | Formulation | | (mL) | (pH) | (%) | (pH) | (%) | (pH) | (%) | (pH) | (%) |
| RT | 2A | (3.2%) | 30 | 7.7 | 96 | 7.1 | 96 | 6.8 | 96 | 6.8 | 93 |
| 5C | 2A | (3.2%) | 30 | 7.7 | 94 | 6.9 | 96 | 7.1 | 96 | 7.0 | 95 |
| RT | 2A | (6.4%) | 18 | 7.6 | 101 | 6.9 | 102 | 6.6 | 102 | 6.6 | 103 |
| 5C | 2A | (6.4%) | 18 | 7.6 | 104 | 7.3 | 104 | 7.0 | 103 | 6.9 | 103 |
| RT | 6 | (3.2%) | 28 | 8.4 | 111 | 7.9 | 111 | 7.9 | 106 | 7.8 | 108 |
| 5C | 6 | (3.2%) | 28 | 8.4 | 108 | 8.1 | 107 | 7.9 | 103 | 7.9 | 105 |
| RT | 6 | (6.4%) | 18 | 8.4 | 108 | 7.9 | 107 | 7.9 | 106 | 7.9 | 108 |
| 5C | 6 | (6.4%) | 18 | 8.4 | 107 | 8.1 | 107 | 7.9 | 106 | 7.9 | 108 |

Substantial changes in appearance or color are not observed.

### Example

Reconstituted Oral Suspension pH Range Testing. The reconstitutable POS formulations described herein are reconstituted by adding water. The pH is measured periodically. The pH range results for Example Formulations 2A with varying amounts of buffer, and at both the 3.2% and 6.4% strengths, are as follows.

| | SOL | Buffer | % w/w | Day 0 | Day 1 | Day 3 | Day 6 | Day 10 |
|---|---|---|---|---|---|---|---|---|
| | (g) | (g) | Buffer | (pH) | (pH) | (pH) | (pH) | (pH) |
| 2A | 1.28 (3.2%) | 0.112 | 1.12 | 8.0 | 6.9 | 6.9 | 6.8 | 6.7 |
| 2A-1 | 1.28 (3.2%) | 0.135 | 1.35 | 8.2 | 7.1 | 7.2 | 7.1 | 7.0 |
| 2A-2 | 1.28 (3.2%) | 0.167 | 1.67 | 8.7 | 7.7 | 7.7 | 7.6 | 8.0 |
| 2A-3 | 1.28 (3.2%) | 0.212 | 2.12 | 9.7 | 8.7 | 8.8 | 8.6 | 8.5 |
| 2A-4 | 1.28 (3.2%) | 0.277 | 2.77 | 10.2 | 9.5 | 9.4 | 9.2 | 9.1 |
| 2A-5 | 1.28 (3.2%) | 0.418 | 4.18 | 10.6 | 10.4 | 10.2 | 10.2 | 10.1 |
| 2A-6 | 1.28 (3.2%) | 0.615 | 6.15 | 10.9 | 10.7 | 10.6 | 10.6 | 10.5 |
| 2A | 2.55 (6.4%) | 0.224 | 1.12 | 8.2 | 6.9 | 6.7 | 6.6 | 6.5 |
| 2A-7 | 2.55 (6.4%) | 0.275 | 1.38 | 8.4 | 7.1 | 6.9 | 6.9 | 6.8 |
| 2A-8 | 2.55 (6.4%) | 0.325 | 1.63 | 8.7 | 7.4 | 7.4 | 7.4 | 7.3 |
| 2A-9 | 2.55 (6.4%) | 0.432 | 2.16 | 9.4 | 8.8 | 8.8 | 8.7 | 8.6 |
| 2A-10 | 2.55 (6.4%) | 0.474 | 2.37 | 9.6 | 9.1 | 9.1 | 9.0 | 8.9 |

### Example

The viscosity of the oral suspension formulations described herein is measured using conventional methods. Without being bound by theory, it is believed herein that the viscosity of the oral suspension can be an important factor in dosing compliance, and in dosage amount consistency.

### Example

The oral suspension formulations described herein are tested for antimicrobial effectiveness, for example according to USP 36, and optionally with different evaluation time frames. The oral suspension formulations described herein are tested for antimicrobial effectiveness for 5, 10, and/or 14 days. For example, the formulations evaluated contain a fixed SOL concentration (for example, 3.2%) in combination with excipients, for example sucrose concentrations at about 10% and about 60%, and for examples potassium sorbate concentrations of about 0.01% and about 0.2%. The oral suspension formulations described herein do not support the growth of bacteria, mold or yeast.

### Example

Solutions/suspensions of the oral suspension formulations described herein are evaluated for stability at various pH levels. For example, SOL at different pH levels (pH 2-10) is incubated at 30° C. for 14 days. Samples are pulled for analysis at Day 7 and Day 14. The limit of total impurities is less than about 4%.

### Example

Threshold bitterness. Compounds described herein are administered to human test subjects at concentrations of 5, 10, 20, 40, and 60 μg/mL in pH 6.0 phosphate buffer. The compounds are compared to equivalent concentrations of azithromycin and clarithromycin. 10 mL of pH 6.0 phosphate buffer is placed in the mouth of the test subjects for 10 seconds, then expectorated. 10 mL of each solution/suspension of SOL, azithromycin, or clarithromycin is placed in the mouth of the test subjects for 10 seconds, then expectorated. The mouth of the test subjects is rinsed with pH 6.0 phosphate buffer. Test subjects report whether the solution/suspension of SOL, azithromycin, or clarithromycin is more bitter than the phosphate buffer alone. Threshold or breakthrough bitterness is the concentration at which the solution/suspension of SOL, azithromycin, or clarithromycin is more bitter than the phosphate buffer alone. SOL exhibits a threshold or breakthrough bitterness that is double that of azithromycin (twice as bitter as azithromycin), but half that of clarithromycin (half as bitter as clarithromycin).

### Example

The flavor profiles of the reconstituted oral suspensions described herein can be measured using conventional methods. (see for example, Keane, P. The Flavor Profile Method. In C. Hootman (Ed.) Manual on Descriptive Testing for Sensory Evaluation. ASTM Manual Series: MNL 13. Baltimore, Md. (1992), the entirety of which is incorporated herein by reference).

### Example

Flavor Profile Method. In an illustrative embodiment, the samples were evaluated using the Flavor Profile Method of descriptive sensory analysis to identify, characterize and quantify the sensory attributes of the reconstituted oral suspensions. The Flavor Profile Method is illustratively described by Keane, “The Flavor profile Method” In C. Hootman (Ed.) Manual on Descriptive Testing for Sensory Evaluation. ASTM Manual Series: MNL 13. Baltimore, Md. (1992). The method can provide a detailed description of the sensory attributes of the suspension, e.g., texture, aroma, taste, mouth-feel. The method includes formal procedures for describing and assessing the aroma (if appropriate) and flavor of a product in a reproducible manner.

Flavor Profile includes measures of the strength or intensity, at which character notes appear, the order in which the character notes appear, a description of all sensations (basic tastes, aromatics, and feeling factors), at specified time intervals after swallowing. Flavor Profile is used to identify the individual attributes of product flavor, including:

Amplitude: Initial overall perception of the balance and fullness of a flavored product, including considering the appropriateness of aromas and flavor notes present, their blend and intensity, and the existence of off-notes. Amplitude scale (in ½ increments): 0-none, 1-low, 2-moderate, 3-high. Initial flavor attributes are generally assessed during the first 10-20 seconds after dosing. An amplitude of about 1½ is generally desirable.

Character notes: aromatics (green stemmy, musty, moldy cardboard, chalky), basic tastes, and feeling factors, each listed in order of appearance and intensity. Intensity scale (in ½ increments): 0-none, 1-slight, 2-moderate, 3-strong. An intensity of about 1 is generally desirable.

Aromatics: volatile components perceived by the olfactory system via the nasopharyngeal passage (retronasal)

Basic tastes: sweet, sour, salty, and bitter Feeling factors: numbing, cooling, warming, burning, drying, oily, astringent, tongue sting, throat burn, trigeminal irritation, and others.

Texture attributes: chalkiness, grittiness, and others.

Aftertaste: measurement of all sensation remaining at selected time intervals. Aftertaste is generally assessed at multiple time points during the 30 minutes after dosing.

Human volunteer panelists evaluate the reconstituted samples. The panelists cleanse their palates with spring water and unsalted crackers. 5 mL of test sample is dispensed into individual 1-ounce plastic cups using a graduated oral syringe and distributed to each panelist. Starting at the same time, the panelists pour the sample directly in to their mouths, swish the contents around the oral cavity for 10 seconds and expectorate. The panelists independently evaluate and record the initial flavor characteristics, then independently evaluate and record the aftertaste characteristics at periodic intervals out to 30 minutes as long as flavor persists. Each sample is optionally tested twice.

### Example

Flavor Leadership Criteria. In another embodiment, formulations described herein are evaluated using Flavor Leadership Criteria, as described in Sjostrom & Cairncross, “What Makes Flavor Leadership?.” Food Technology 2(7):56-58 (1953). Flavor Leadership Criteria include having a quickly recognizable identifying flavor, developing full flavor that rapidly blends with and covers the active and base characteristics, having no unpleasant feeling factors, having no off-notes in the early impression or in the aftertaste, and having a short (or appropriate) aftertaste.

### Example

Flavor Profile Results. Reconstitutable POS formulations 1, 2, and 3 are evaluated and compared to a control formulation (6.40 g SOL, 0.10 g tribasic sodium phosphate, qs 100 mL water). Reconstitutable POS formulations 1, 2, and 3 are obtained in a prescription bottle and transferred to a 100 mL-graduated cylinder. Water is added to the graduated cylinder to bring the volume to approximately 90 mL, and the graduated cylinder is agitated and inspected to ensure the powder is wet and in suspension. The volume is brought up to 100 mL and the suspension is returned to the prescription bottle, which is recapped and agitated by hand for three minutes. Human volunteer panelists evaluate the reconstituted samples in duplicate.

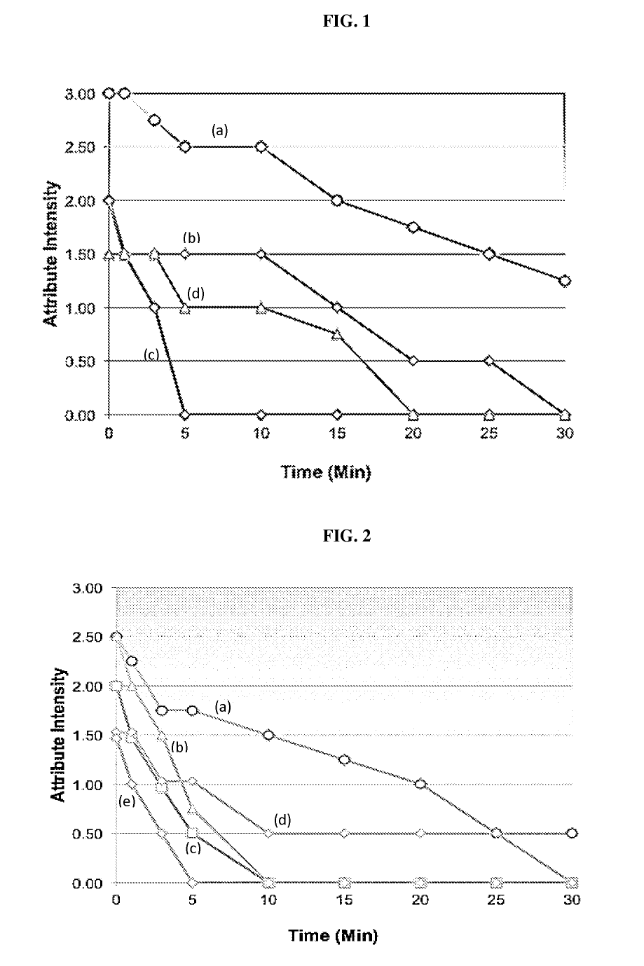

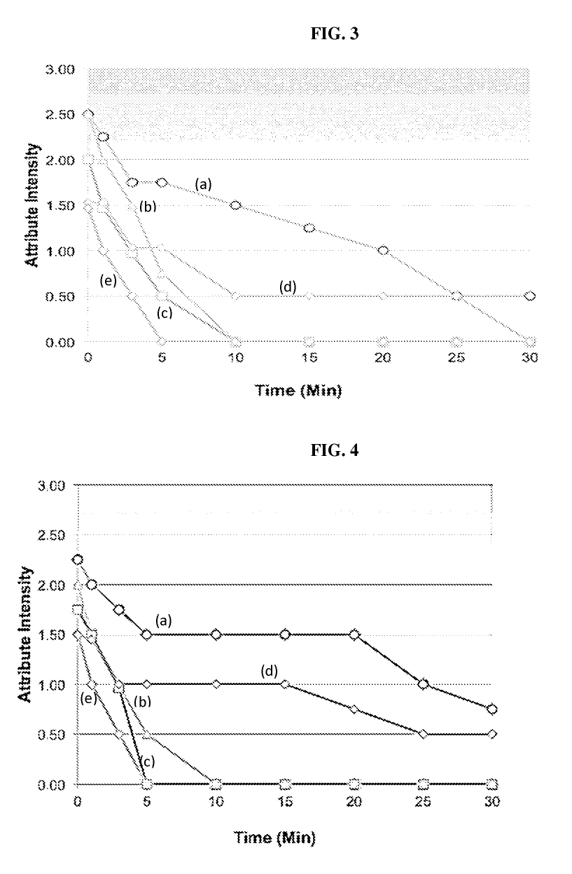

Referring to FIG. 1, FIG. 2, FIG. 3, and FIG. 4, it was observed that the oral suspension formulations prepared from each of the reconstitutable POS formulations 1, 2, and 3 were superior to the control formulation in each of the criteria of undesirable taste attributes, including bitterness and aromatics (green stemmy and moldy cardboard) taste attributes. It was also observed that the binary sweeteners (POS formulations 1 and 2) were unexpectedly superior to the single sweeteners (POS formulation 3) at various concentrations. In each instance, the undesirable taste attributes fall below about 1½ (the typical patient perception level) faster for each of POS formulations 1, 2, and 3 compared to the control formulation.

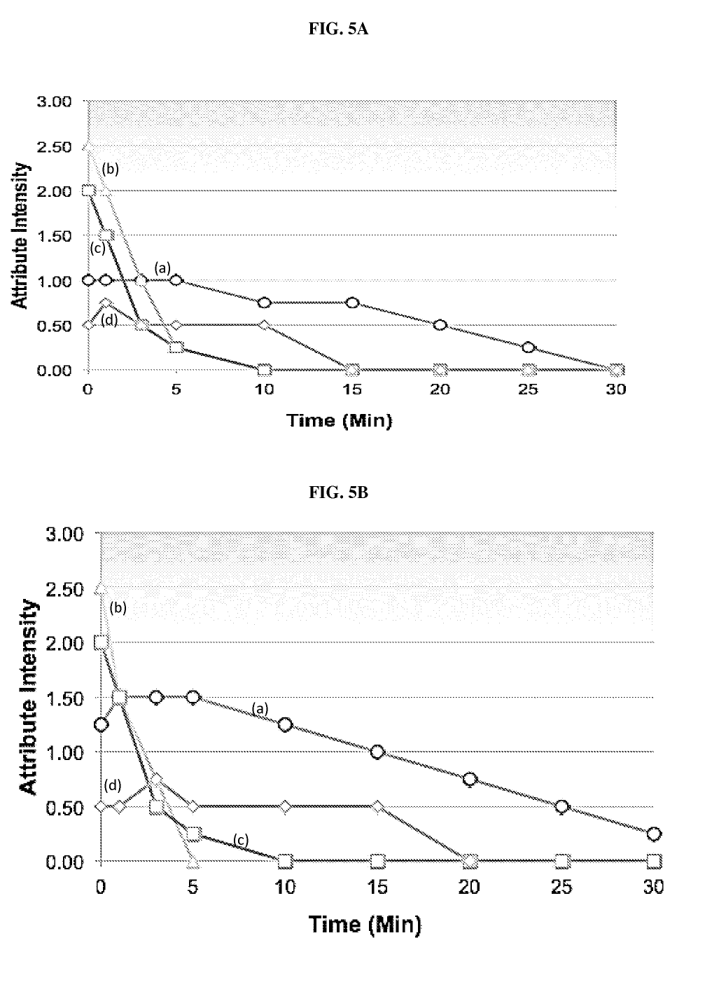

Referring to FIG. 5A, it was observed that sucrose, aspartame, and Magnasweet reconstitutable POS formulations 11 and 13 (data not shown) further reduced bitterness compared to the control formulation. In addition, it was observed that the flavor was stable over the entire 7-day observation period, as shown in FIG. 5B.

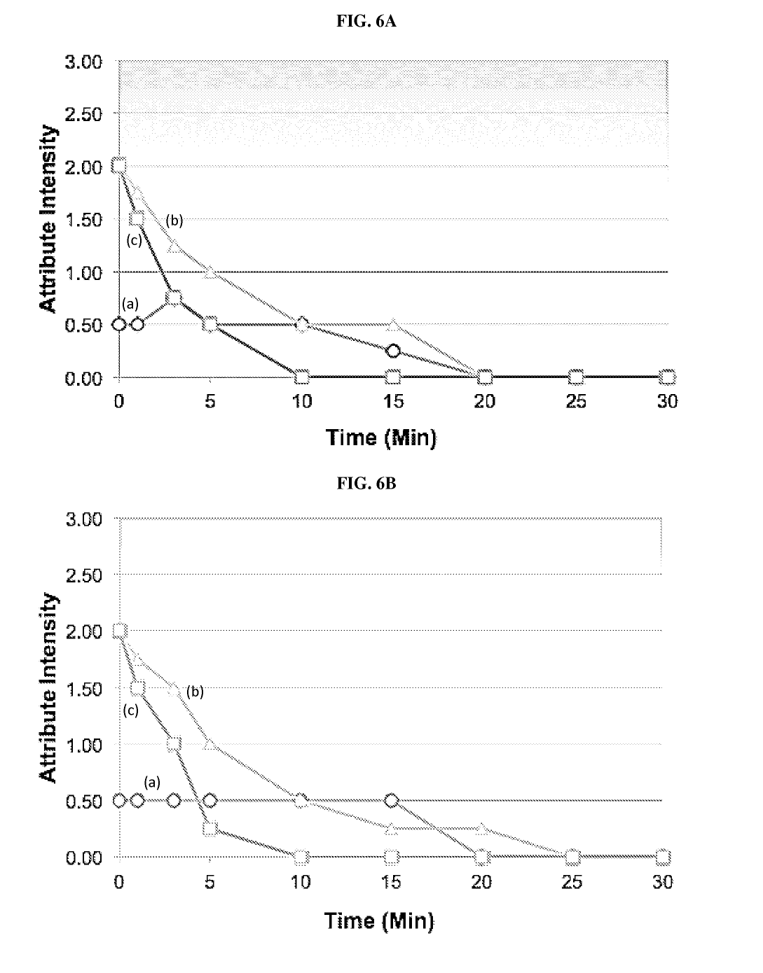

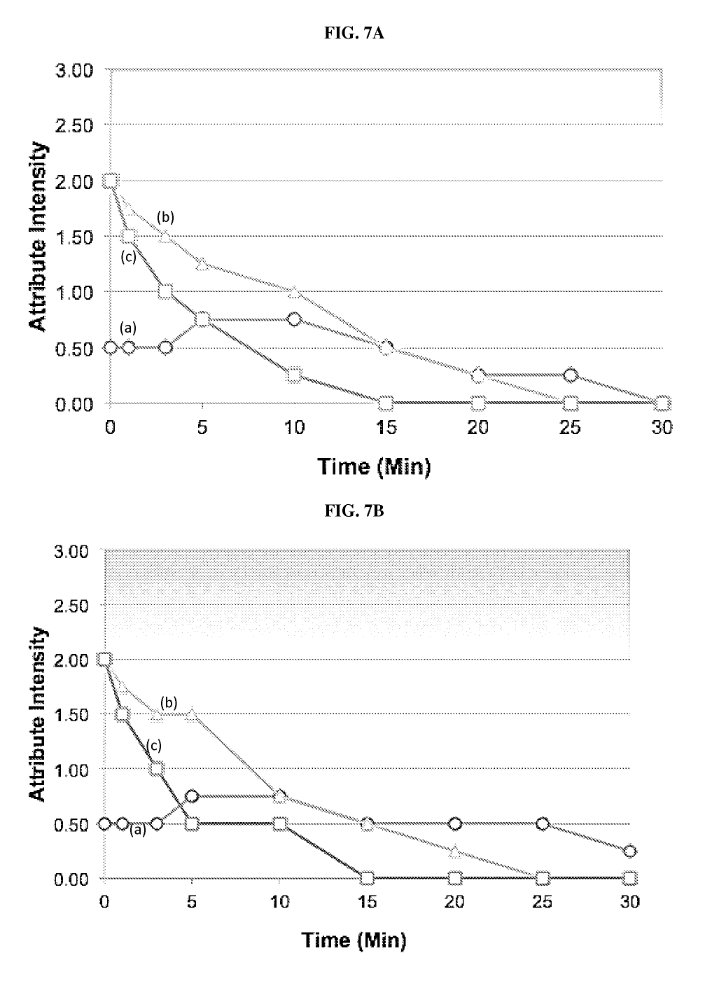

Referring to FIG. 6A and FIG. 7A, it was observed that sucrose, sucralose, and NaCl reconstitutable POS formulations 12 and 14 further reduced bitterness compared to the control formulation. In addition, it was observed that the flavor was stable over the entire 7-day observation period, as shown in FIG. 6B and FIG. 7B.

### Example

Bioavailability and pharmacokinetics of oral suspension formulation. The oral suspension formulations described herein are administered to humans once daily at a dose that delivers 400 mg of SOL. The Cₘₐₓ, Tₘₐₓ, and total exposure (area under curve, AUC) are measured and compared to once daily oral administration of 400 mg capsules or 400 mg tablets of SOL. Dosing is continued for 5, 10, and/or 14 days. The Cₘₐₓ, Tₘₐₓ, and AUC for the oral suspension formulations described herein are comparable to the tablets and capsules.

1. A composition in the form of a reconstituable powder for oral suspension, the composition comprising one or more compounds of the formula 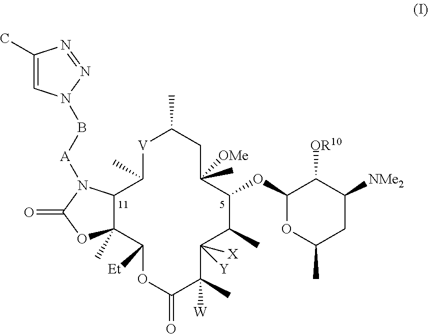
or salts thereof, or hydrates thereof, or combinations thereof, wherein:
R¹⁰ is hydrogen or acyl;
X is H; and Y is OR⁷; where R⁷ is a monosaccharide or disaccharide, alkyl, heteroalkyl, cycloalkyl, cycloheteroalkyl, aryl, heteroaryl, acyl, or C(O)—NR⁸R⁹, where R⁸ and R⁹ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl, or R⁸ and R⁹ are taken together with the attached nitrogen to form a heterocycle; or X and Y are taken together with the attached carbon to form carbonyl;
V is C(O), C(═NR¹¹), CH(NR¹², R¹³), or N(R¹⁴)CH₂; where R¹¹ is hydroxy or alkoxy, R¹² and R¹³ are each independently selected from the group consisting of hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl; and R¹⁴ is hydrogen, hydroxy, alkoxy, alkyl, heteroalkyl, cycloalkyl, cycloheteroalkyl, aryl, heteroaryl, arylalkyl, heteroarylalkyl, acyl, sulfonyl, ureido, and carbamoyl;
W is H, F, Cl, Br, I, or OH;
A is CH₂, C(O), C(O)O, C(O)NH, S(O)₂, S(O)₂NH, C(O)NHS(O)₂;
B is C₀-C₁₀ alkylene, C₂-C₁₀ alkenylene, or C₂-C₁₀ alkynylene; and
C is hydrogen, hydroxy, acyl, acyloxy, sulfonyl, ureido, or carbamoyl, or alkyl, heteroalkyl, cycloalkyl, cycloheteroalkyl, aryl, heteroaryl, arylalkyl, or heteroarylalkyl, each of which is optionally substituted.

2. The composition of claim 1 wherein R¹⁰ is hydrogen.

3. The composition of claim 1 wherein X and Y are taken together with the attached carbon to form carbonyl.

4. The composition of claim 1 wherein V is C(O).

5. The composition of claim 1 wherein the compound is of the formula 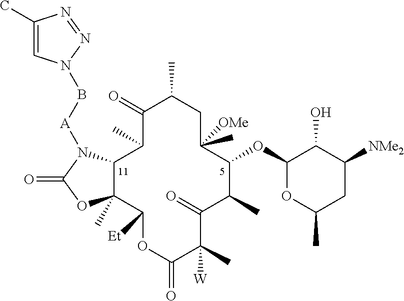
or a salt or a hydrate thereof.

6. The composition of claim 1 wherein W is H or F.

7. The composition of claim 1 wherein A is CH₂.

8. The composition of claim 1 wherein B is (CH₂)ₙ where n is an integer ranging from 0-10; and

9. The composition of claim 1 wherein C is aryl, heteroaryl, arylalkyl, or heteroarylalkyl, each of which is optionally substituted.

10. The composition of claim 1 wherein the compound is of the formula 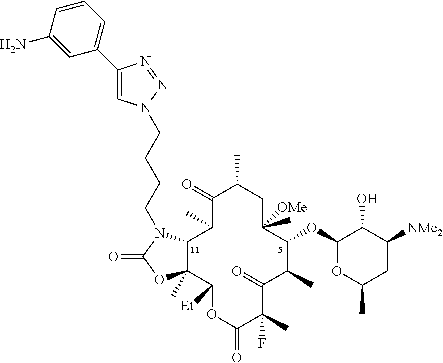 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C]
or a salt or a hydrate thereof.

11. The composition of claim 1 wherein the compound is of the formula 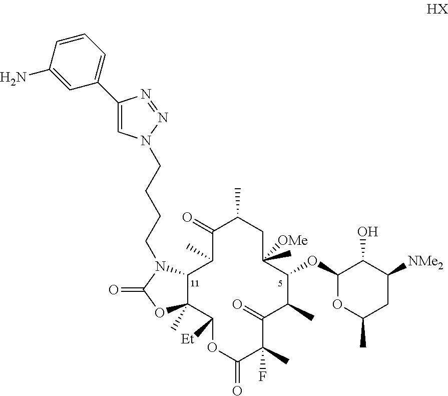 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C]
where HX is a pharmaceutically acceptable acid.

12. The composition of claim 1 wherein the compound is of the formula

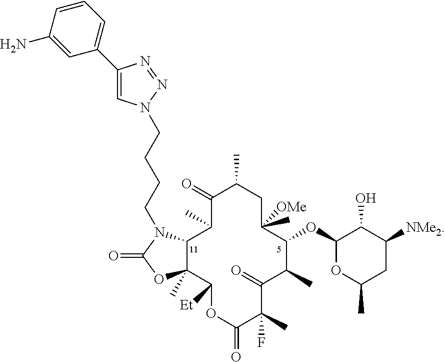 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C]

13. A kit comprising the composition of claim 1 and instructions for reconstituting the composition to prepare an oral suspension formulation; and optionally a container for reconstituting.

14. A pharmaceutical formulation comprising the composition of claim 1 and water.

15. (canceled)

16. The formulation of claim 14 in the form of a suspension.

17. The formulation of claim 14 wherein the bitterness is at a threshold index of about 1.5 or less.

18. The formulation of claim 14 wherein the compound is soluble in the formulation at a level of about 1 mg/mL or less.

19. The composition of claim 1 further comprising one or more pharmaceutically acceptable constituents selected from the group consisting of suspending agents, sweeteners, preservatives, surfactants, flavoring agents, and combinations thereof.

20. The composition of claim 1 further comprising a binary sweetener.

21. The composition of claim 1 further comprising sucrose and aspartame.
